(19) United States
(12) Patent Application Publication
(10) Pub. No.: US 2011/0091443 A1
(43) Pub. Date: Apr. 21, 2011

(54) HEPARIN-CONJUGATED FIBRIN GEL AND METHOD AND KIT FOR PREPARING SAME

(75) Inventors: Byung-Soo Kim, Seoul (KR); O-Ju Jeon, Seoul (KR); Hee-Seok Yang, Seoul (KR)

(73) Assignee: Industry-University Cooperation Foundation Hanyang University

(21) Appl. No.: 12/990,121

(22) PCT Filed: Apr. 28, 2009
(86) PCT No.: PCT/KR09/02220

§ 371 Date: Dec. 21, 2010

(30) Foreign Application Priority Data
Apr. 28, 2008 (KR) 10-2008-0039322
Apr. 24, 2009 (KR) 10-2009-0035779

(51) Int. Cl. A61K 38/48; A61K 38/18; A61P 17/02; A61P 19/08; A61P 9/10

(52) U.S. Cl. 424/94.64; 514/7.6

(57) ABSTRACT

A method of preparing a heparin-conjugated fibrin gel is provided, which includes activating heparin, conjugating the activated heparin with fibrinogen to prepare heparin-conjugated fibrinogen, mixing free fibrinogen with the heparin-conjugated fibrinogen to prepare a fibrinogen mixture, and mixing thrombin with the fibrinogen mixture. In addition, a heparin-conjugated fibrin gel prepared by the above method and a kit for preparing the same are provided. According to the method of preparing the heparin-conjugated fibrin gel, the heparin-conjugated fibrin gel having an affinity for drugs such as growth factors may be easily prepared at low costs, and can also be used as a therapeutic drug excellently effective on generation of tissues such as bones, skin, blood vessels, cartilages, etc. by sustainably releasing drugs such as growth factors to a local site for a long period of time through injection into a human body.

### TECHNICAL FIELD

This application claims the benefit of Korean Patent Application No. 2008-39322, filed on Apr. 28, 2008 and 2009-35779 filed on Apr. 24, 2009, the disclosure of each of which is hereby incorporated herein by reference in its entirety.

The present invention relates to tissue engineering focusing on repair of damaged tissue and organs, and more particularly, to regeneration of tissues associated with transferring growth factors.

Specifically, a method of preparing a fibrin gel conjugated with injectable heparin containing a growth factor is provided, in which activated heparin conjugated with fibrinogen is mixed with free fibrinogen and dissolved, which is then mixed with thrombin, thereby completing an injectable heparin-conjugated fibrin gel.

Meanwhile, a growth factor having an affinity for heparin physically binds to the heparin-conjugated fibrin gel prepared according to the above-mentioned method of the present invention and is injected into a human body, thereby resulting in providing sustained release of growth factors for a long period of time. Therefore, it can be used as a therapeutic drug for stimulating regeneration of tissue, such as bones, skin, blood vessels, cartilages, etc.

### BACKGROUND ART

Tissue engineering is a research field focusing on repair of tissues and organs damaged due to diseases or accidents. Methods of using an injectable gel as a matrix for regenerating tissues are being widely researched, and may reduce recovery time, pain and costs imposed on patients since a drug can be easily injected into a target site with no surgical procedures. Examples of the injectable gels used so far include collagen gels, fibrin gels and alginic acid gels. Particularly, since the fibrin gel can be obtained from a patients blood, it does not bring about an immune reaction. Therefore, it is receiving great attention as an autologous matrix for tissue engineering.

Fibrin is a kind of scleroprotein, which is non-soluble protein generated by reaction of fibrinogen in a plasma and an enzyme, thrombin. That is, fibrin is prepared in a gel type by reaction with fibrinogen in the presence of thrombin at room temperature through enzymatic polymerization. The fibrin gel prepared as such is significant to tissue engineering focusing on repair of damaged tissues and organs. Particularly, the fibrin gel facilitates delivery of growth factors, and therefore various researches into fibrin gels are being conducted to regenerate tissue such as bones, skin, blood vessels, cartilages, etc.

Since heparin, a kind of glycosaminoglycan, contains a large quantity of sulfate groups, thereby being highly negative-charged, it is often used as an affinity ligand conjugated with specific heparin-conjugated protein. The characteristic of the affinity ligand is also applied to chromatography for purifying protein. However, recent attention has focused on applying this characteristic to fix heparin-conjugated protein to a carrier of a protein drug to specifically deliver to a specific site in a body.

### DISCLOSURE OF INVENTION

### Technical Solution

The present invention is directed to a simple method of preparing a fibrin gel directly conjugated with heparin, thereby having long-term, sustained release of a contained drug.

The present invention is also directed to a protein carrier including a fibrin gel directly conjugated with heparin, thereby having long-term, sustained release of a contained drug, and a kit for preparing a fibrin gel as described above.

To this end, the present inventors have conducted research into a fibrin gel composition capable of containing a large quantity of growth factors without using a peptide, thereby resulting in preparation of fibrinogen directly conjugated with heparin, which is activated before conjugation.

In addition, it was found that a fibrin gel could not easily be obtained by crosslinking polymerization of heparin-directly conjugated fibrinogens only, but a fibrin gel allowing excellent sustained release of a contained drug and easy handling could be obtained by crosslinking polymerization of both fibrinogens directly conjugated with heparins and free fibrinogens, which are not conjugated with heparins.

Accordingly, in one aspect of the present invention, a method of preparing a heparin-conjugated fibrin gel is provided, which includes: activating heparin; conjugating the activated heparin with fibrinogen to prepare heparin-conjugated fibrinogen; mixing the heparin-conjugated fibrinogen prepared in the previous step with free fibrinogen to prepare a fibrinogen mixture; and mixing the fibrinogen mixture prepared in the previous step with thrombin.

A kit for preparing a heparin-conjugated fibrin gel, including heparin-conjugated fibrinogen, free fibrinogen which is not conjugated with heparin, and thrombin, and a growth factor carrier including a fibrin gel containing fibrinogen directly conjugated with heparin and a growth factor are provided.

According to the method of preparing the heparin-conjugated fibrin gel, the heparin-conjugated fibrin gel having an affinity for drugs such as growth factors may be easily prepared at low costs, and can also be used as a therapeutic drug excellently effective on generation of tissues such as bones, skin, blood vessels, cartilages, etc. by sustainably releasing drugs such as growth factors to a local site for a long period of time through injection into a human body.

### BRIEF DESCRIPTION OF DRAWINGS

These and/or other objects, aspects and advantages of the invention will become apparent and more readily appreciated from the following description of the exemplary embodiments, taken in conjunction with the accompanying drawings of which:

FIG. 1 shows NMR data obtained to confirm whether activated heparin is conjugated with fibrinogen;

FIG. 2 shows FTIR data obtained to confirm whether activated heparin is conjugated with fibrinogen;

FIG. 3 is a graph showing a release behavior of bone morphogenetic protein-2 (BMP-2) in an injectable heparin-conjugated fibrin gel composition;

FIG. 4 is a photograph showing formation of gels using mixtures of heparin-free fibrinogen or heparin-conjugated fibrinogen with thrombin solutions through crosslinking polymerization, respectively;

FIG. 5 is photographs showing formation of gels according to increasing contents of heparin-free fibrinogen; and

FIG. 6 is a graph showing a release behavior of platelet-derived growth factor (PDGF) in an injectable heparin-conjugated PRP fibrin gel composition.

### BEST MODE FOR CARRYING OUT THE INVENTION

Hereinafter, the present invention will be described in more detail with reference to exemplary embodiments.

The present invention provides a method of preparing a heparin-conjugated fibrin gel, which includes: activating heparin; conjugating the activated heparin with fibrinogen to prepare heparin-conjugated fibrinogen; mixing the heparin-conjugated fibrinogen prepared in the previous step with free fibrinogen to prepare a fibrinogen mixture; and mixing the fibrinogen mixture prepared in the previous step with thrombin. The fibrin gel prepared according to the present invention may be an injectable fibrin gel.

According to the method of preparing a heparin-conjugated fibrin gel of the present invention, the fibrinogen may be derived from a mammal, and preferably, human plasma.

The term ‘heparin-conjugated fibrinogen’ denotes fibrinogen conjugated with heparin prepared by activation of heparin described herein, and the term ‘free fibrinogen’ or ‘fibrinogen’ individually used herein denotes fibrinogen which is not conjugated with other compounds, particularly, heparin.

The heparin used in the present method may have a low molecular weight, which may be in the range of 1000 to 20000.

Activation of the heparin in the method of preparing a fibrin gel according to the present invention is to introduce a NHS-group to the heparin, which may be conducted by any method. While the activation of the heparin may be performed by reaction of the heparin, and carbodiimide and N-hydroxysuccinimide, and activated into NHS-heparin, the present invention is not limited thereto.

Preparation of the heparin-conjugated fibrinogen in the method of preparing a fibrin gel according to the present invention is accomplished by reaction between the NHS-heparin and fibrinogen. Specifically, the heparin-conjugated fibrinogen is prepared by reaction between a carboxy group of the NHS-heparin and an amino group present in protein of the fibrinogen. Meanwhile, the heparin-conjugated fibrinogen prepared by the above-mentioned reaction may be readily used for the subsequent step, or stored in a liquid or dry powder type for future use depending on its purpose. Preferably, the heparin-conjugated fibrinogen may be lyophilized and easily stored.

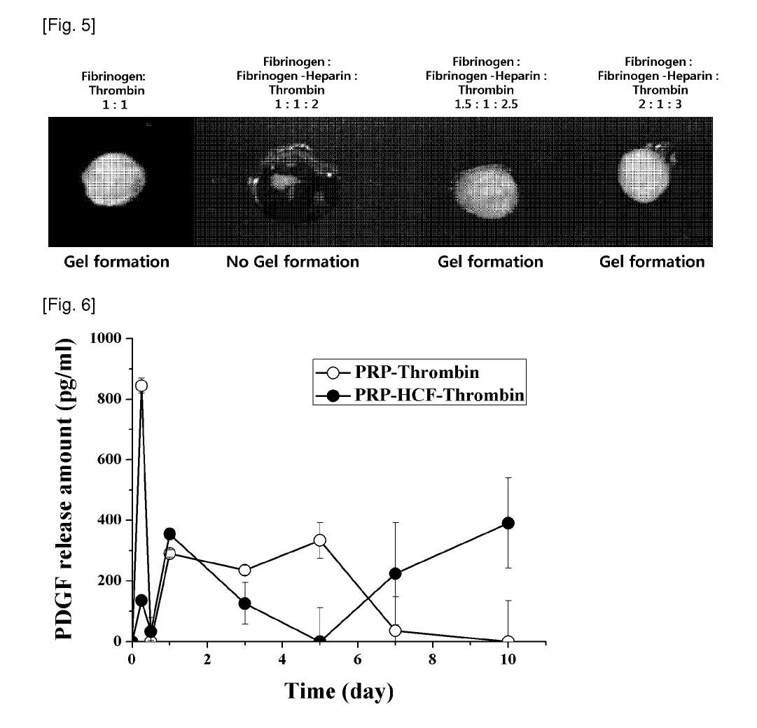

During the preparation of the fibrinogen mixture in the method of preparing the fibrinogen gel according to the present invention, a ratio of free fibrinogen to heparin-conjugated fibrinogen may be 1:1 to 5:1. Within this range, clot formation of a fibrin gel may be controlled. When the ratio is less than 1:1, a mechanical property of the gel is poor, and it is difficult to form a fibrin gel (refer to FIG. 5). If the gel is not formed, extended-release, which is an object of the present invention, may not be provided, and the gel having a very poor mechanical property may not provide long-term sustained release due to a large quantity of initial burst even when it is injected into a human body. On the other hand, when the ratio is greater than 5:1, the gel is well formed, but a relative amount of heparin mixed with fibrinogen in the fibrin gel decreases, and thus a quantity of growth factors conjugated therewith also decreases. As a result, the fibrin gel used to contain a therapeutically-effective dose of a protein drug increases in quantity, and efficiency in sustained-release of the protein drug drops.

In one embodiment, a kit for preparing a heparin-conjugated fibrin gel including heparin-conjugated fibrinogen, heparin-free fibrinogen and thrombin is provided. A ratio of the heparin-conjugated fibrinogen to the heparin-free fibrinogen in the kit may be 1:1 to 5:1. The heparin-conjugated fibrinogen, the heparin-free fibrinogen and the thrombin may be individually lyophilized and stored, and each of them may be dissolved in a buffer solution and then mixed together before preparing a gel according to the preparation method of the present invention. Preferably, components each dissolved in a buffer solution are easily mixed using a two-way syringe just before preparing a fibrin gel.

In another embodiment, a kit for preparing a heparin-conjugated fibrin gel is provided, including a growth factor conjugated with heparin in addition to heparin-conjugated fibrinogen, free fibrinogen not conjugated with heparin and thrombin. The growth factor may be selected from, but is not limited to, the group consisting of BMPs, vascular endothelial growth factors (VEGFs), transforming growth factors-β (TGFs-β), platelet-derived growth factors (PDGFs) and fibroblast growth factors (FGFs). In the present invention, at least one growth factor may be included, which may be, for example, a mixture created by mixing various kinds of growth factors such as a platelet-rich plasma (PRP).

How to control additional components or reaction conditions for preparing fibrin are well known to those skilled in the art. For example, to dissolve the fibrinogen mixture, an aprotinin solution may be used. Aprotinin serves to delay natural enzymatic decomposition of fibrin when the fibrin is prepared by crosslinking polymerization of fibrinogen and thrombin. In the present method, the thrombin may be dissolved in a solution containing calcium chloride. Depending on concentrations of the fibrinogen and thrombin solutions, a clot-formation time and clot firmness may be controlled. In this case, the quantity of dissolved thrombin may be the same as that of the fibrinogen mixture.

In another embodiment of the present invention, a growth factor carrier including a fibrin gel having heparin-directly conjugated fibrinogen, and at least one growth factor is provided. The fibrin gel prepared according to the present invention is bonded with the above-mentioned growth factor, and injected into a human body in the form of a fibrin gel composition, thereby releasing growth factors to a local site for a long period of time. Therefore, it can be used as a therapeutic drug stimulating regeneration of tissue such as bones, skin, blood vessels, cartilages, etc. Specifically, the fibrin gel composition prepared by the method of the present invention may be widely applied to treat bone defects, burns, diabetic or ischemic foot ulcers, ischemic heat diseases, limb ischemia, degenerative cartilage diseases, etc. through simple injection into a human body.

### MODE FOR THE INVENTION

### Example 1

### Activation of Heparin

100 mg (0.00002 mole) of low-molecular weight heparin (molecular weight: 4000-6000; Sigma) was completely dissolved in 1 ml of 2-(N-morpholino)ethane sulfonic acid (MES, Sigma) buffer solution (0.05M, pH 6.0), and 0.0046 g of N-hydroxysuccinimide (NHS, 0.00008M) and 0.015336 g of N-(3-dimethylaminopropyl)-N-ethylcarbodiimide hydrochloride (EDC, 0.004M) were added thereto for 12-hour reaction at 4° C., thereby resulting in preparing an NHS-heparin solution. The NHS-heparin solution was precipitated and extracted using acetone anhydride, and then lyophilized for 24 hours.

### Example 1-1

### Conjugation of the Activated Heparin with Fibrinogen

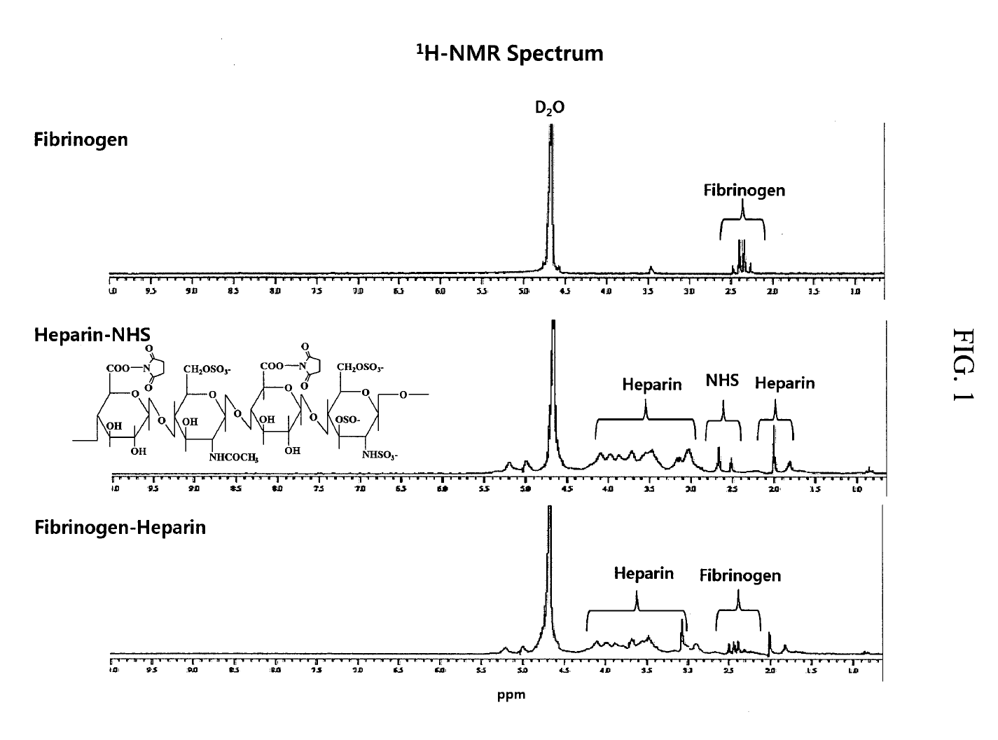

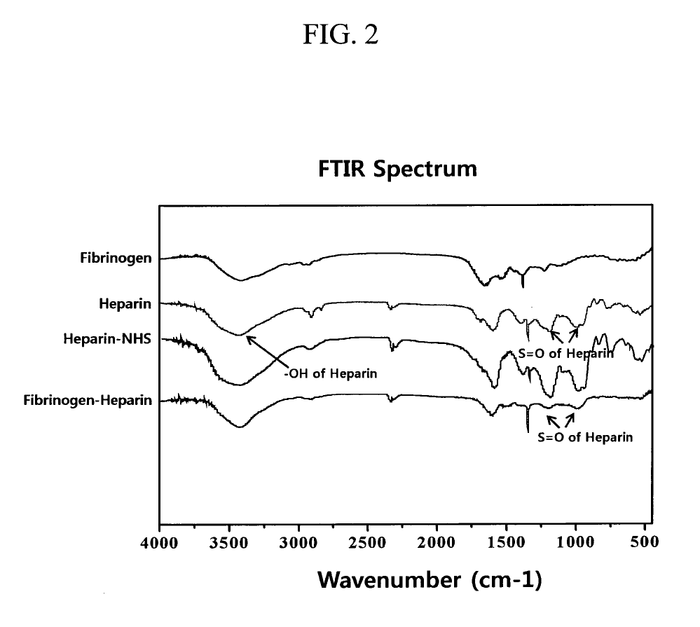

The lyophilized NHS-heparin (60 mg) prepared in Example 1 and human plasma-derived fibrinogen (100 mg) were completely dissolved in 20 ml of phosphate buffered saline (pH 7.4), and reacted for 3 hours at 4° C. Here, the fibrinogen was slowly dissolved so as not to bubble. After the reaction, the resulting solution was precipitated using acetone anhydride and lyophilized in a dark room, thereby preparing heparin-conjugated fibrinogen. Conjugation of the activated heparin with fibrinogen was confirmed through NMR and FTIR analyses, and the analysis results are shown in FIGS. 1 and 2, respectively. Meanwhile, the heparin-conjugated fibrinogen prepared as described above was completely dissolved in 20 ml of phosphate buffered saline, and residual, unreacted heparin was eluted by dialyzing through a porous membrane bag (MWCO: 12-14000) for 24 hours at 4° C. while changing distilled water more than three times. The eluted heparin-conjugated fibrinogen was lyophilized for 48 hours in a dark room, thereby resulting in white powder of heparin-conjugated fibrinogen.

### Example 1-2

### Quantification of Heparin in Heparin-Conjugated Fibrinogen

Heparin in heparin-conjugated fibrinogen prepared in Example 1-1 was quantified by toluidine blue analysis. Briefly, the heparin-conjugated fibrinogen was added to 2 ml of 0.02% NaCl aqueous solution contained in a test tube, and then 5000 of 0.005% toluidine blue-contained 0.001N HCl solution was added. The above mixture was incubated for 30 minutes, and gently shaken. 3 ml of n-hexane was added to the mixture and vigorously shaken for 10 to 15 seconds, followed by settling the mixture to stabilize layers. When removal of bubbles was confirmed, thereby stably separating an upper layer of hexane and a lower layer of water, about 2000 of sample was taken from the lower layer of water to subject to ELISA. Then, the sample was analyzed using a UV spectrophotometer at 630 nm. The content of heparin was calculated as 42.73 mg/g.

### Example 2

### Preparation of Fibrin Gel Containing Bone Morphogenetic Protein

Bone morphogenetic protein (BMP) was dissolved in an aprotinin solution used as a protease inhibitor. The heparin-conjugated fibrinogen (33 mg) prepared in Example 1-1 was mixed with human plasma-derived fibrinogen (66 mg) commercially available at a ratio of 1:2, and dissolved in the above-mentioned aprotinin solution (1000 μl), thereby preparing a fibrinogen solution. Meanwhile, an equivalent amount of human plasma-derived thrombin (10 mg) was dissolved in a calcium chloride-dissolved solution (1000 μl), and mixed with the fibrinogen mixture solution, resulting in a BMP-conjugated fibrin gel (100 μl) protein carrier.

### Example 2-1

### Release Behavior of BMP in Fibrin Gel

Control group 1 was created by artificially mixing BMP with a heparin-free fibrin gel commercially available, and Control group 2 was created by mixing heparin and BMP with a fibrin gel commercially available. That is, in Control groups 1 and 2, the BMPs were conjugated with none or one of the fibrin gel and heparin.

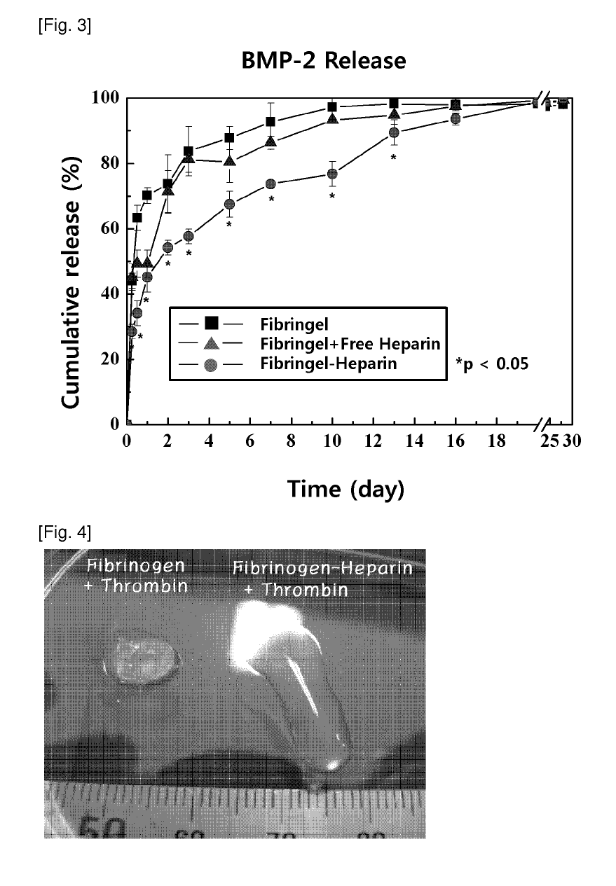

Meanwhile, the BMP-conjugated fibrin gel composition prepared in Example 2 as an experimental group was slowly stirred at 37° C., and the BMP release behavior was examined for 28 days. The BMP release behavior is shown in the graph of FIG. 3. As shown in FIG. 3, it could be confirmed that the about 80% or more BMP was released from the fibrin gel compositions of control groups 1 and 2 in the first three days, whereas about 80% or more BMP was slowly released from the fibrin gel composition of the experimental group (Example 2 of the present invention), which was conjugated with heparin, for 13 days.

### Example 3

### Evaluation of Effect of Free Fibrinogen and Heparin Content on Preparation of Fibrin Gel

Whether heparin-conjugated fibrinogen was individually capable of preparing a fibrin gel by the action of thrombin was examined.

Without mixing heparin-free fibrinogen, heparin-conjugated fibrinogen was prepared according to Example 1, and examined in degree of forming a gel using fibrin preparation conditions including thrombin. When a mixture of fibrinogen in an aprotinin buffer solution and a thrombin solution was polymerized using only heparin-free fibrinogen through crosslinking, gel formation was facilitated, but when the mixture was polymerized with only heparin-conjugated fibrinogen through crosslinking, a gel was not formed (Refer to FIG. 4). It could be confirmed in FIG. 4 that even when a formation plate was inclined after the gel forming reaction, the product using the heparin-free fibrinogen did not run, whereas the product using the heparin-conjugated fibrinogen could not make a gel, and thereby ran.

Afterwards, fibrin gels were prepared by mixing fibrinogens which were not conjugated with heparins before initiation of reactions (free fibrinogens) and heparin-conjugated fibrinogens at ratios of 1:1, 1.5:1 and 2:1, respectively. In FIG. 5, gels formed according to increasing contents of the heparin-free fibrinogen were shown. The formation of the gel was facilitated as the content of the heparin-free fibrinogen was increased.

### Example 4

### Release Behavior of Platelet-Derived Growth Factor in Fibrin Gel

1 ml of platelet-rich plasma (PRP) was mixed with 100 mg of fibrinogen mixture prepared by mixing the heparin-conjugated fibrinogen prepared in Example 1-1 and human plasma-derived fibrinogen commercially available at a ratio of 1:2, and human-plasma derived thrombin (10 mg) was dissolved in a calcium chloride-dissolved solution (1000 μl) and mixed with the fibrinogen mixture solution, resulting in 100 μl of PRP-conjugated fibrin gel protein carrier.

As a control group, a fibrin gel protein carrier prepared as described above except for using a heparin-free fibrin gel, which is commercially available, was used.

A PRP-thrombin fibrin gel and a PRP-heparin-conjugated fibrinogen (HCF) prepared in vitro, as experimental groups, were slowly stirred at 37° C., and a release behavior of a platelet-derived growth factor (PDGF) was examined for 10 days. The release behavior of the PDGF is shown in the graph of FIG. 6. As shown in FIG. 6, it could be confirmed that the PDGF was mostly released from the control group fibrin gel during day 1 to day 6, whereas the PDGF was sustainably released from the experimental group, which was the PDGF-heparin-conjugated fibrin gel composition even on day 10 on a higher level than day 1.

According to a method of preparing a heparin-conjugated fibrin gel, a fibrin gel conjugated with heparin, which has an affinity for a drug such as a growth factor, can be easily prepared at low costs.

In addition, a heparin-conjugated fibrin gel prepared by the present method can be prepared as a fibrin gel composition containing a growth factor by binding the growth factor having an affinity for heparin. The fibrin gel composition prepared by the present method can effectively deliver a growth factor, and can thus be developed at low costs as a highly effective therapeutic drug capable of efficiently treating bone defects, burns, diabetic or ischemic foot ulcers, ischemic heat diseases, limb ischemia, and degenerative cartilage diseases through injection into a human body by sustainably releasing drugs such as growth factors, etc. to a local site for a long period of time.

While exemplary embodiments of the present invention have been shown and described, it will be appreciated by those skilled in the art that various changes can be made to the described exemplary embodiments without departing from the spirit and scope of the invention defined by the claims and their equivalents.

1. A method of preparing a heparin-conjugated fibrin gel, comprising:
a) activating heparin;
b) conjugating the activated heparin with fibrinogen to prepare heparin-conjugated fibrinogen;
c) mixing free fibrinogen with the heparin-conjugated fibrinogen in step b) to prepare a fibrinogen mixture; and
mixing thrombin with the fibrinogen mixture in step c).

2. The method according to claim 1, wherein the fibrin gel is an injectable gel.

3. The method according to claim 1, wherein the fibrinogen is mammal-derived fibrinogen.

4. The method according to claim 3, wherein the mammal is human.

5. The method according to claim 1, wherein the heparin has a molecular weight of about 1000 to about 20000.

6. The method according to claim 1, wherein the heparin is activated into NHS-heparin by reaction between heparin, carbodiimide and N-hydroxysuccinimide in step a).

7. The method according to claim 1, wherein the heparin-conjugated fibrinogen in step b) is prepared by the reaction between a carboxyl group of NHS-heparin and an amino group present in protein of the fibrinogen.

8. The method according to claim 1, wherein the fibrinogen mixture in step C is prepared by mixing the fibrinogen with the heparin-conjugated fibrinogen at a ratio of 1:1 to 5:1.

9. A kit for preparing a heparin-conjugated fibrin gel, comprising:
heparin-conjugated fibrinogen;
free fibrinogen which is not conjugated with heparin; and
thrombin.

10. The kit according to claim 9, further comprising a growth factor conjugated with heparin.

11. The kit according to claim 9, wherein the growth factor includes at least one selected from the group consisting of bone morphogenetic proteins (BMPs), vascular endothelial growth factors (VEGFs), transforming growth factors-β (TGFs-β), platelet-derived growth factors (PDGFs) and fibroblast growth factors (FGFs).

12. A growth factor carrier comprising a fibrin gel including fibrinogen directly conjugated with heparin and at least one growth factor.
